(19) United States
(12) Patent Application Publication
(10) Pub. No.: US 2024/0358763 A1
(43) Pub. Date: Oct. 31, 2024

(54) SUPPLEMENT FOR HUMAN MILK FROM ALLERGIC WOMEN

(71) Applicant: N.V. Nutricia, Zoetermeer (NL)

(72) Inventors: Jon Genuneit, Markranstädt (DE); Linda Siziba, Karlsruhe (DE); Dietrich Rothenbacher, Pfaffenhofen-Kadeltshofen (DE); Lisa Buchenauer, Leipzig (DE); Marko Mank, Utrecht (NL); Bernd Stahl, Utrecht (NL); Selma Wiertsema, Utrecht (NL); Joris Henricus van Sadelhoff, Veenendaal (NL); Gerrit Witte, Utrecht (NL); Astrid Hogenkamp, Utrecht (NL)

(21) Appl. No.: 18/566,549

(22) PCT Filed: May 31, 2022
(86) PCT No.: PCT/EP2022/064731

§ 371 Date: Dec. 1, 2023

(30) Foreign Application Priority Data
Jun. 2, 2021 (EP) 21177471.6

(51) Int. Cl. A61K 35/20; A23L 33/00; A61K 9/00; A61K 31/198; A61P 37/08

(52) U.S. Cl. CPC A61K 35/20; A23L 33/40; A61K 9/0095; A61K 31/198; A61P 37/08

(57) ABSTRACT

The present invention relates to a method of fortifying human nutrition with free amide containing amino acids, a method of providing infant nutrition to an infant of a lactating woman that suffers from allergy or of a lactating woman that has suffered from allergy, and supplements or fortified infant formulas comprising free L-glutamine and/or free L-asparagine for use in the treatment or prevention of allergy in infants of a lactating woman that suffers from allergy or of a lactating woman that has suffered from allergy.

### FIELD OF THE INVENTION

Field of the invention is the field of supplements or fortifiers for human milk and infant formulas, in particular for human milk from allergic mothers.

### STATE OF THE ART

Human milk (HM) is the preferred source of early life nutrition. It has been shown to support healthy infant growth and development and to provide protection against several immunological conditions in early life, including various infectious and allergic diseases. Various bioactive components in human milk have been identified that could contribute to these unique abilities, including several proteins and peptides. Besides peptides and proteins, HM contains free amino acids (FAAs). FAAs account for 5-10% of the total amino acid (TAA) content in HM.

Although breast milk is the preferred nutrition for infants, there are maternal conditions where the breast milk composition is less optimal. An example is the milk from diabetic or obese mothers, that was found to be lower in levels of long chain polyunsaturated fatty acids (LC-PUFA) (Jackson et al 1994, Am J Clin Nut. 60:353-361). Another example is milk from mothers from preterm babies. Special human milk fortifiers have been developed to fortify this milk with additional ingredients, in particular protein and minerals, to enable the especially high needs for these nutrients by preterm infants. WO 2013/1058524 discloses glutamine-based supplement in the form of a powder. This supplement is intended for preterm or low birth weight infants and has a positive effect on brain development. EP 0 665 012 is directed to an antiallergy agent comprising glutamine as active agent, wherein the agent is suitable for use in nutritional compositions for infants and is useful for prevention of the induction of allergic diseases in infancy, such as atopic dermatitis and infantile asthma, and the treatment thereof.

However, little is known about the milk of allergic mothers. In the study of Hettinga et al. (2015, PLOS ONE, 2015, DOI: 10.1371/journal.pone.0122234) a difference in the breast milk proteome between allergic and non-allergic mothers was identified.

### SUMMARY OF THE INVENTION

The inventors have found that the concentration of free amide containing amino acids, asparagine and glutamine, and in particular glutamine, is significantly lower in the milk of mothers that were allergic or had a history of allergy. The levels of these free amide containing amino acids was furthermore associated with the health outcome in the infants in symptoms associated with allergy, such as food allergy and asthma.

In light of this finding the inventors have developed a supplement comprising free amide containing amino acids that can be used to fortify the human milk of lactating women that are allergic or have a history of allergy. Alternatively, an infant formula has been developed that is enriched in the free amide containing amino acids for use in feeding infants that are at least partly breast-fed by a lactating woman that is allergic or by a lactating woman has a history of allergy, in particular by compensating for the deficit in the concentration of the free amide containing amino acids L-asparagine and/or L-glutamine in the human milk from a lactating woman that suffers from allergy or from a lactating woman that has suffered from allergy.

### DETAILED DESCRIPTION

### Free Amide Containing Amino Acids

The present invention relates to supplements for infant nutrition, in particular milk fortifiers, and to fortified infant formulas, in each case comprising free amide containing amino acids, L-asparagine and/or L-glutamine, in particular in unbound, that means in free form. Preferably, the supplements for infant nutrition, in particular milk fortifiers, or the fortified infant formulas comprise at least L-glutamine, in particular free L-glutamine, more preferably L-glutamine and L-asparagine, in particular free L-glutamine and free L-asparagine. Preferably, the supplements for infant nutrition, in particular milk fortifiers, or the fortified infant formulas comprise free amide containing amino acids consisting of L-glutamine. The supplements for infant nutrition, in particular milk fortifiers, or fortified infant formulas according to the present invention are therefore particularly intended for use in infants that are completely or partially breast-fed by lactating women that suffer from allergy or by lactating women that have a history of allergy, such as, but not limited to hay fever, atopic dermatitis or asthma. It was surprisingly found that the milk of lactating mothers suffering from allergy or of lactating mothers having a history of allergy comprises reduced levels of the free amide containing amino acids L-glutamine and L-asparagine, in particular reduced levels of L-glutamine, when compared to women that do not suffer from or have a history of allergy.

Glutamine is a conditionally essential AA in the neonatal period and is known to have effects on the growth and function of the intestines and to modify immune responses in a favourable manner in neonatal animals, including effects that may be beneficial in terms of preventing neonatal allergies and infections ((Van den Berg et al, 2007, Arch Ped & Adol Med 161:1095-1101, Sadelhoff et al, 2020, Front. Immunol., https://doi.org/10.3389/fimmu.2020.01007). Thus, a lower intake of free L-glutamine via human milk by infants of mothers with an history in allergic disease may have health implications on the infants and increase the risk for allergy and/or infection. L-asparagine can become a conditionally essential amino acid when levels of L-glutamine decline. Furthermore, the inventors found that a correlation existed between the levels of free L-asparagine and allergy related symptoms. L-glutamine and L-asparagine both have a characteristic carboxamide side chain and can be classified as free amide containing amino acids. Both are polar, aliphatic amino acids and sometimes have a similar biological function. It was found that the level of free L-asparagine in the milk inversely correlated with the occurrence of allergy. In particular, there was an inverse correlation with asthma at 6 years. L-glutamine and L-asparagine are commercially available. An example of a suitable suppliers of these ingredients is Ajinomoto (Japan).

The present invention is particularly directed to the prevention of allergy in infants of mothers that suffer from allergy or of mothers that have a history of allergy, in particular to the prevention of allergy in infants that are completely or partially breast-fed by a lactating mother that suffers from allergy or by a lactating mother that has a history of allergy. More preferably, the present invention is directed to the prevention of allergy later in life, more preferably asthma later in life. Later in life refers to a period after breast feeding.

In one aspect, the present invention pertains to a method of fortifying infant nutrition, in particular human milk from a lactating woman that suffers from allergy or from a lactating woman that has suffered from allergy or an infant formula, said method comprising the steps of:
a) providing a supplement comprising L-glutamine and/or L-asparagine, in particular free L-glutamine and/or free L-asparagine, and
b) adding the supplement provided in step a) to infant nutrition, in particular human milk from a woman that suffers from allergy or from a woman that has a history of suffering from allergy or an infant formula, thereby obtaining a fortified infant nutrition, in particular a fortified human milk or a fortified infant formula.

In a preferred embodiment of the present invention, the fortified infant nutrition, in particular the fortified human milk or the fortified infant formula, obtained in step b) is in a subsequent step c) administered, in particular fed, to an infant, preferably to an infant of a lactating woman that suffers from allergy or of a lactating woman that has suffered from allergy, in particular to an infant who is at least partially fed with human milk of a lactating woman that suffers from allergy or of a lactating woman that has suffered from allergy.

The present invention is further directed to a method of providing infant nutrition to an infant of a lactating woman that suffers from allergy or of a lactating woman that has suffered from allergy, in particular to an infant who is at least partially fed with human milk of a lactating woman that suffers from allergy or of a lactating woman that has suffered from allergy, said method comprising the steps of:
i) fortifying infant nutrition with a supplement comprising L-glutamine and/or L-asparagine, in particular free L-glutamine and/or free L-asparagine, and
ii) feeding the fortified infant nutrition to the infant.

In a preferred embodiment of the present invention, the infant nutrition is a) human milk from a lactating woman that suffers from allergy or from a lactating woman that has suffered from allergy or b) an infant formula. Accordingly, the fortified infant nutrition is preferably either a) human milk from a lactating woman that suffers from allergy or from a lactating woman that has suffered from allergy fortified with a supplement comprising L-glutamine and/or L-asparagine, in particular free L-glutamine and/or free L-asparagine, or b) an infant formula fortified with a supplement comprising L-glutamine and/or L-asparagine, in particular free L-glutamine and/or free L-asparagine.

In a preferred embodiment of the present invention, the supplement comprising L-glutamine and/or L-asparagine, in particular free L-glutamine and/or free L-asparagine, is a milk fortifier. Preferably, the supplement comprising L-glutamine and/or L-asparagine, in particular free L-glutamine and/or free L-asparagine, is a supplement for infant formulas.

Preferably, the supplement comprising L-glutamine and/or L-asparagine, in particular free L-glutamine and/or free L-asparagine, or the infant formula fortified with a supplement comprising L-glutamine and/or L-asparagine, in particular free L-glutamine and/or free L-asparagine, is in powder form.

In a preferred embodiment of the present invention, the fortified infant nutrition contains L-glutamine and/or L-asparagine, in particular free L-glutamine and/or free L-asparagine, in an amount of at least 8.5 mg/100 ml, preferably at least 9 mg/100 ml, preferably, preferably at least 10 mg/100 ml, preferably at least 15 mg/100 ml, preferably at least 20 mg/100 ml, preferably at least 25 mg/100 ml, preferably at least 30 mg/100 ml, preferably at least 40 mg/100 ml, preferably at least 50 mg/100 ml. Preferably, the fortified infant nutrition contains L-glutamine and/or L-asparagine, in particular free L-glutamine and/or free L-asparagine, in an amount of at most 800 mg/100 ml, preferably at most 500 mg/100 ml, preferably at most 250 mg/100 ml, preferably at most 100 mg/100 ml, preferably at most 80 mg/100 ml, preferably at most 60 mg/100 ml, preferably at most 40 mg/100 ml.

The invention also pertains to the use of a supplement comprising L-glutamine and/or L-asparagine, in particular free L-glutamine and/or free L-asparagine, for fortifying infant nutrition of an infant of a lactating woman that suffers from allergy or of a lactating woman that has suffered from allergy, in particular an infant who is at least partially fed with human milk of a lactating woman that suffers from allergy or of a lactating woman that has suffered from allergy.

In a preferred embodiment of the present invention, the supplement used for fortifying infant nutrition of an infant of a lactating woman that suffers from allergy or of a lactating woman that has suffered from allergy provides the infant nutrition with L-glutamine and/or L-asparagine, in particular free L-glutamine and/or free L-asparagine, in an amount of at least 8.5 mg/100 ml, preferably at least 9 mg/100 ml, preferably, preferably at least 10 mg/100 ml, preferably at least 15 mg/100 ml, preferably at least 20 mg/100 ml, preferably at least 25 mg/100 ml, preferably at least 30 mg/100 ml, preferably at least 40 mg/100 ml, preferably at least 50 mg/100 ml. Preferably, the milk fortifier used for fortifying infant nutrition of an infant of a lactating woman that suffers from allergy or of a lactating woman that has suffered from allergy provides the infant nutrition with L-glutamine and/or L-asparagine, in particular free L-glutamine and/or free L-asparagine, in an amount of at most 800 mg/100 ml, preferably at most 500 mg/100 ml, preferably at most 250 mg/100 ml, preferably at most 100 mg/100 ml, preferably at most 80 mg/100 ml, preferably at most 60 mg/100 ml, preferably at most 40 mg/100 ml.

Particularly preferred, the infant nutrition fortified with the supplement comprising L-glutamine and/or L-asparagine, in particular free L-glutamine and/or free L-asparagine, is human milk from a lactating woman that suffers from allergy or from a lactating woman that has suffered from allergy. In a further preferred embodiment, the infant nutrition fortified with the supplement comprising L-glutamine and/or L-asparagine, in particular free L-glutamine and/or free L-asparagine, is an infant formula.

The present invention is also directed to a supplement comprising L-glutamine and/or L-asparagine, in particular free L-glutamine and/or free L-asparagine, preferably at least L-glutamine, in particular free L-glutamine, in particular to a supplement comprising L-glutamine and/or L-asparagine, in particular free L-glutamine and/or free L-asparagine, preferably at least L-glutamine, in particular free L-glutamine, for use in the treatment or prevention of allergy, in particular for use in the treatment or prevention or reduction of the risk of allergy, in an infant of a lactating woman that suffers from allergy or of a lactating woman that has suffered from allergy, in particular wherein the infant is at least partially fed with the human milk of the lactating woman. Preferably, the supplement is packed in unit dose, e.g. in the form of sachets. In a preferred embodiment, each sachet contains the amount of L-glutamine and/or L-asparagine, in particular free L-glutamine and/or free L-asparagine, to supplement 100 ml of infant formula or expressed human milk. Preferably, one sachet contains at least 8 mg L-glutamine, in particular free L-glutamine, more preferably at least 20 mg L-glutamine, in particular free L-glutamine. Preferably, L-asparagine, in particular free L-asparagine, is present in the fortifier in an amount of 0.8 to 2 mg per sachet. The L-glutamine and/or L-asparagine, in particular free L-glutamine and/or free L-asparagine, are preferably mixed with a carrier, such as maltodextrin or starch, in order to facilitate an accurate dosing. For example, the sachet may contain 500 mg of which 25 mg L-glutamine, in particular free L-glutamine, 5 mg L-asparagine, in particular free L-asparagine, and the remainder 470 mg maltodextrin or starch. The maximal amount of the sum of L-glutamine and L-asparagine, in particular free L-glutamine and/or free L-asparagine, is preferably 500 mg per sachet.

The supplement can particularly be used to fortify infant nutrition, in particular expressed human milk from mothers that suffer from allergy or from mothers that have a history of allergy or an infant formula. In a preferred embodiment of the present invention, the fortified infant nutrition, in particular the fortified expressed human milk from mothers that suffer from allergy or from mothers that have a history of allergy or the fortified infant formula, is administered to the infant at least once a day, preferably at least twice a day.

In a particular embodiment, the supplement comprising L-glutamine and/or L-asparagine, in particular free L-glutamine and/or free L-asparagine, can also be used to fortify standard infant formula, in particular standard infant formula that is intended for infants that are at least partially breast-fed by lactating women suffering from allergy or by lactating women having a history of allergy. Partially breast-fed infants are defined as infants receiving at least once a day breast feeding and at least once a day a feeding with infant formula.

Standard infant formula, based on intact cow's milk protein, contains very low levels of free amino acids, in particular very low levels of free L-glutamine and L-asparagine compared to human milk (see Chuang et al., 2005, JPGN 40:496-500, and Agostoni et al., 2000, Journal of the American College of Nutrition, Vol. 19, No. 4, 434-438).

The present invention therefore also pertains to an infant formula fortified with L-glutamine and/or L-asparagine, in particular free L-glutamine and/or free L-asparagine, preferably at least L-glutamine, in particular free L-glutamine, in particular to an infant formula fortified with L-glutamine and/or L-asparagine, in particular free L-glutamine and/or free L-asparagine, preferably at least L-glutamine, in particular free L-glutamine, for use in the treatment or prevention of allergy, in particular for use in the treatment or prevention or reduction of the risk of allergy, in an infant of a lactating woman that suffers from allergy or of a lactating woman that has suffered from allergy, in particular wherein the infant is at least partially fed with the human milk of the lactating woman.

Preferably, the infant formula, in particular the infant formula for use in the treatment or prevention of allergy, in particular for use in the treatment or prevention or reduction of the risk of allergy, in an infant of a lactating woman that suffers from allergy or of lactating woman that has suffered from allergy, comprises L-glutamine and/or L-asparagine, in particular free L-glutamine and/or free L-asparagine, in an amount of at least 8.5 mg/100 ml, preferably at least 9 mg/100 ml, preferably, preferably at least 10 mg/100 ml, preferably at least 15 mg/100 ml, preferably at least 20 mg/100 ml, preferably at least 25 mg/100 ml, preferably at least 30 mg/100 ml, preferably at least 40 mg/100 ml, preferably at least 50 mg/100 ml, when the infant formula is in liquid form or reconstituted in water from a powder to a ready-to-drink formula.

In a preferred embodiment of the present invention, the infant formula, in particular the infant formula for use in the treatment or prevention of allergy, in particular for use in the treatment or prevention or reduction of the risk of allergy, in an infant of a lactating woman that suffers from allergy or of lactating woman that has suffered from allergy, comprises L-glutamine and/or L-asparagine, in particular free L-glutamine and/or free L-asparagine, in an amount of at most 800 mg/100 ml, preferably at most 500 mg/100 ml, preferably at most 250 mg/100 ml, preferably at most 100 mg/100 ml, preferably at most 80 mg/100 ml, preferably at most 60 mg/100 ml, preferably at most 40 mg/100 ml.

The amount of L-glutamine, in particular free L-glutamine, in the fortified infant formula is preferably at least 8 mg/100 ml, more preferably at least 15 mg/100 ml, when the infant formula is in liquid form or reconstituted in water from a powder to a ready-to-drink formula. The amount of the sum of L-glutamine and L-asparagine, in particular free L-glutamine and free L-asparagine, in the fortified infant formula is preferably not more than 800 mg/100 ml, more preferably not more than 100 mg/100 ml, when the infant formula is in liquid form or reconstituted in water from a powder to a ready-to-drink formula. The amount of L-glutamine, in particular free L-glutamine, in the fortified infant formula is preferably not more than 800 mg/100 ml, more preferably not more than 100 mg/100 ml when the infant formula is in liquid form or reconstituted in water from a powder to a ready-to-drink formula.

In a particularly preferred embodiment of the present invention, the fortified infant nutrition, in particular the fortified human milk or the fortified infant formula, comprises L-glutamine and L-asparagine, in particular free L-glutamine and free L-asparagine, in a weight ratio of 50/1 to 5/1, preferably 45/1 to 6/1, more preferably 40/1 to 7/1.

Preferably, the supplement or the infant nutrition, in particular the supplement or the infant formula for use in the treatment or prevention of allergy, in particular for use in the treatment or prevention or reduction of the risk of allergy, in an infant of a lactating woman that suffers from allergy or of a lactating woman that has suffered from allergy, comprises L-glutamine and L-asparagine, in particular free L-glutamine and free L-asparagine, in a weight ratio of 50/1 to 5/1, preferably 45/1 to 6/1, more preferably 40/1 to 7/1. This ratio reflects the ratio as observed in milk from non-allergic mothers.

Preferably, at least once a day, preferably at least twice a day the fortified infant nutrition, in particular the fortified human milk or the fortified infant formula, is administered to the infant.

In a further preferred embodiment of the present invention, the supplement comprising L-glutamine and/or L-asparagine, in particular free L-glutamine and free L-asparagine, or the fortified infant formula is in powder form. L-glutamine and L-asparagine have a limited stability in aqueous solutions. When in powder form, and reconstituted with water to a ready-to-drink form and when the infant formula is consumed within 2 hours of reconstitution, or within 24 hours when refrigerated, according the general recommendation for handling infant formulas, the L-glutamine and/or L-asparagine degradation is limited.

Preferably, the infant is a term infant. Preferably, the infant has a birth weight appropriate for gestational age. Preferably, the infant is a term infant and has a birth weight appropriate for gestational age. In the context of the present invention, infants are defined as human subjects with an age of 0 to 12 months. Preferably, the infant has an age of 3 to 12 month, more preferably an age of 6 to 12 months. Atopic dermatitis, the first onset in the atopic march typically first appears after 3 to 6 months of age.

Preferably, the infant is at risk of allergy. Infants at risk for allergy particularly benefit from the invention. In a further preferred embodiment of the present invention, the allergy, in particular the allergy the lactating mother is suffering from or has suffered from, is selected from hay fever, atopic dermatitis and asthma.

In the context of the present invention, the term “a” is meant to include the meaning of “one” or “one or more”.

In the context of the present invention, the term “comprising” preferably has the meaning of “containing” or “including” meaning that the composition in question at least comprises the specifically identified component without excluding the presence of further components. However, in a preferred embodiment the term comprising is also understood to have the meaning of “consisting essentially of” and in a most preferred embodiment of “consisting”. The term “consisting essentially of” excludes the presence of substantial amounts of further components except the specifically identified component of the composition. The term “consisting” excludes the presence of any further compound, no matter in which quantity in the composition identified.

Further preferred embodiments of the invention are subject of the subclaims.

The present invention is illustrated by way of the following examples and the corresponding figure.

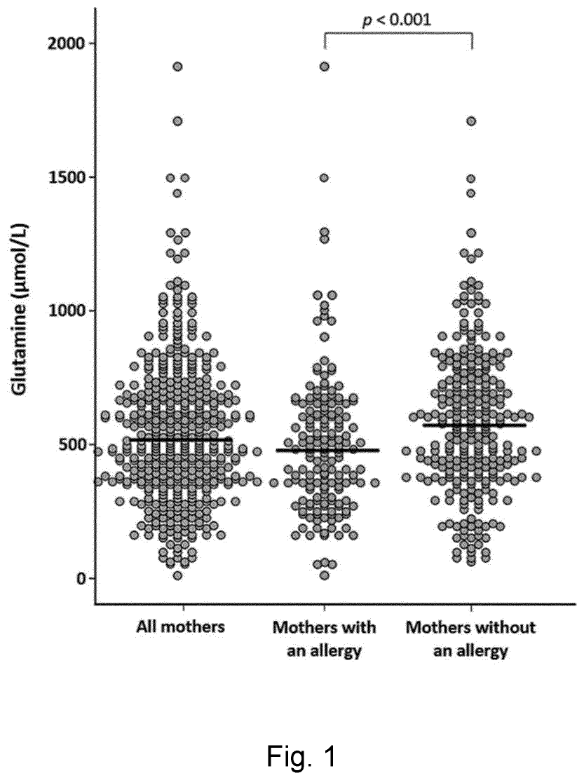

FIG. 1 shows the level of free L-glutamine in 6-month human milk samples from all mothers (n=441), from mothers with an allergy or a history of allergy (n=167), and mothers without an allergy (n=274).

### EXAMPLE 1: BREAST MILK ANALYSIS STUDY: BREAST MILK FROM ALLERGIC MOTHERS HAS A REDUCED CONTENT OF FREE GLUTAMINE AND ASPARAGINE

### Study Design and Study Population

Data were obtained from the Ulm SPATZ Health Study, an ongoing birth cohort study including 970 mothers and their 1006 newborns. Details are described in Logan, C., et al., Pediatrics, 2016. 137: p. e20154473. Mothers were recruited from the general population in the period 04/2012-05/2013. Exclusion criteria were outpatient delivery, maternal age <18 years, transfer of the newborn or the mother to intensive care immediately after delivery, and/or insufficient knowledge of the German language. For the purpose of the present study, the population was further restricted to singleton full term (gestational age ≥37 weeks) births. The study was approved by the ethics board of Ulm University (No. 311/11) and all mothers gave written informed consent before participating.

### Human Milk (HM) Sample Collection

HM samples were collected at approximately 6 months post-delivery. Mothers were instructed to manually express or pump the first ˜10 mL of HM (i.e. foremilk) between 9 am and 12 pm, after breakfast and before lunch but at least one-hour after the last feeding. In rare instances, trained study nurses helped mothers with expression. Mothers stored the HM samples in the refrigerator until study nurses collected the samples and delivered them to the study centre.

### Amino Acid Analyses

HM samples were stored at −80° C. until analysis of AAs. Levels of FAAs (i.e. unbound AAs) and TAAs (i.e. unbound+conjugated AAs) were analysed by liquid chromatography as described in detail in Sadelhoff, J. H. J., et al., Nutrients, 2018. 10 (9) 1233; doi: 10.3390/nu10091233. This method disabled the detection of proline and cysteine, yielding a total of 18 detectable FAAs and taurine, ornithine and citrulline. The measurements of TAAs required acidic hydrolysis of proteins, which enabled the detection of 15 TAAs and disabled the detection of tryptophan, cysteine, and proline. The acidic hydrolysis process also transformed asparagine into aspartate and glutamine into glutamate, disabling the detection of these TAAs individually. All measurements were done in the same lab and by the same technician.

### Categorization of Mothers

Maternal allergy was self-reported and classified as mothers who reported a history of hay fever, atopic dermatitis or asthma. Non-allergic mothers reported the absence of an allergic disease.

### Infant Immune Outcome

A reported doctor's diagnosis of wheeze in the past 12 months was assessed at 1 year, 2 years and at each yearly follow-up by separate self-administered questionnaires from parents. Wheezing phenotypes were classified into three categories: transient (report of wheeze at age 1 but not at age 2 and 3), persistent (report of wheeze at age 2 but not age 1 and 3) and late onset (report of wheeze at age 1, 2 and 3). Atopic dermatitis (AD) status was assessed by separate parent and paediatrician reports of doctor-diagnosed AD assessed at 1, 2 and 3 years post-delivery. AD was analysed separately as “parent-only”, “paediatrician-only” and “both parent and paediatrician” reported AD diagnosis. Children with at least one positive report of AD were defined as cases in the relevant outcome category. A reported doctor's diagnosis of food allergy in the past 12 months was assessed at 1 year of age by separate self-administered questionnaires from parents and the children's primary care paediatricians. Lower respiratory tract infections (including pneumonia, bronchitis, pertussis, tracheobronchitis, Krupp, bronchiolitis and flu) at 1 year of age were established by questionnaires completed by the children's primary care paediatricians. Asthma was assessed at 6 years of age.

### Statistical Analyses

Data were checked for normal distribution using Shapiro Wilk test and visual inspection of histogram plots. Maternal factors that associated with levels of any FAA or TAA 6-month HM were included as confounders in the partial correlation tests. Differences in AA levels in HM between mothers with or without a history in allergy (either active or non-active) were assessed by Welch's t-tests.

Modified Poisson regression analyses were conducted to estimate the effects of FAA levels in HM on infant outcomes of wheeze, atopic dermatitis, lower respiratory tract infections, food allergy and asthma. Risk ratios (RR) were modelled with individual FAA levels as continuous independent variable. Models were adjusted for all maternal (age, education, BMI, parity and history of smoking), infant (sex, birth weight and gestational age) and birth (delivery mode)-related putative confounders associated with ≥5% change in crude estimate of the effect of any individual FAA on any infant health outcome.

### Maternal and Infant Characteristics

HM samples for AA analyses were available from 441 lactating women at 6 months of lactation. At this timepoint, 167 mothers (37.7%) were reported to be allergic or have a history of allergy.

### The AA Composition in HM at 6 Months of Lactation and the Maternal Determinants

It was tested whether maternal history in allergic disease influences AA levels in HM. Levels of free glutamine were significantly lower in 6-month HM of mothers with a history in allergic disease (mean difference=81.1 μM, p=0.002) (see FIG. 1). This difference remained significant after adjustment for pre-pregnancy BMI, maternal age and infant sex (p=0.006). No differences were found for any of the other FAAs or TAAs (Table 1). Also, the level of asparagine was lower in breast milk of allergic mothers, when compared to milk of non-allergic mothers (p=0.1). No differences were observed in the sum of all FAA, or all TAA.

TABLE 1
FAA levels in HM of mothers with and
without history in allergic disease

| | Mean (SD) | | |
|---|---|---|---|
| | | No history | History in |
| FAAs | Total mothers | in allergy | allergy |
| (mg/100 ml) | (n = 441) | (n = 274) | (n = 167) |
| Asparagine | 0.233 (0.113) | 0.240 (0.113) | 0.224 (0.114)* |
| Glutamine | 8.02 (3.92) | 8.47 (3.92) | 7.28 (3.85)** |
| All FAAs | 45.64 (9.14) | 45.90 (9.23) | 45.21 (9.00) |
| All TAAs | 765.4 (117.8) | 762.8 (110.7) | 769.6 (127.6) |

FAA: free amino acid; TAA: total amino acids. Amounts are expressed as mg/100 ml/l
**p < 0.05 when compared with mothers with no history of allergy,
*p = 0.1 when compared with mothers with no history of allergy.

Of all the other FAA tested either the difference observed between the milk from allergic or non-allergic mothers was not statistically different, or the amino acid level was slightly higher in the milk from allergic mothers (data not shown).

### Associations of FAAs in HM and Infant Health Outcomes

Modified Poisson Regression revealed a significant association between free asparagine and asthma. Asthma was assessed at 6 years of life and a significant negative association was found with the level of free asparagine in the human milk 0.78 (RR 95% CI 0.68; 0.90, p<0.05). Together with the known positive effects of L-glutamine on immune and intestinal epithelial cells, resulting in positive effects on preventing infection, inflammation and allergy, this is indicative for a preventive effect on allergy, including asthma later in life, by these amino acids.

### EXAMPLE 2: MILK FORTIFIER FOR INFANTS PARTIALLY OR COMPLETELY BREAST-FED BY ALLERGIC MOTHERS OR MOTHERS WITH A HISTORY OF ALLERGY

A milk fortifier A is packet in a sachet. The milk fortifier A is in powder form and comprises about 50 mg L-glutamine per sachet, and the remainder, up to 500 mg, being a suitable carrier like maltodextrin.

A milk fortifier B is packet in a sachet. The milk fortifier B is in powder form and comprises about 50 mg L-glutamine per sachet and 5 mg of L-asparagine, and the remainder, up to 500 mg, being a suitable carrier like maltodextrin.

### EXAMPLE 3: METHOD FOR SUPPLEMENTING HUMAN MILK FROM A LACTATING WOMAN THAT SUFFERS FROM ALLERGY OR HAS A HISTORY OF ALLERGY

Milk is expressed by a lactating woman that is allergic or has a history of allergy.

One sachet of the milk fortifier supplement A or B of example 2 is added to the expressed milk, and the fortified human milk is fed to the infant, preferably at least once a day.

### EXAMPLE 4: METHOD FOR SUPPLEMENTING AN INFANT FORMULA FOR AND INFANT PARTIALLY BREAST-FED BY A LACTATING WOMAN THAT SUFFERS FROM ALLERGY OR HAS A HISTORY OF ALLERGY

A standard infant formula containing intact cow's milk protein (whey protein/casein in 6/4 weight ratio, 2.0 g protein per 100 kcal, about 1.35 g protein per 100 ml ready to drink formula) is prepared in a bottle. One sachet of the milk fortifier supplement A or B of example 2 is added to the bottle, mixed with the infant formula and fed to the infant, preferably at least once a day.

### EXAMPLE 5: SUPPLEMENTED INFANT FORMULA FOR AN INFANT PARTIALLY BREAST FED BY A LACTATING WOMAN THAT SUFFERS FROM ALLERGY OR HAS A HISTORY OF ALLERGY

Standard infant formula is manufactured as known in the art, in compliance with international directives for infant formula. The protein component is intact protein derived from cow's milk protein (whey protein concentrate and skim milk powder) and contains whey protein/casein in a 6/4 weight ratio. The amount of protein is 2.0 g/100 kcal. The infant formula is in powder form and contains 276 kcal/100 gram. When the powder is reconstituted to a ready-to-drink formula the amount of kcal per 100 ml is 66.

Free L-glutamine is added to this powdered infant formula in an amount of 75 mg/100 g powder. Without addition of free L-glutamine the amount of L-glutamine would have been below 3.75 mg per 100 g powder in accordance with the levels found for Nutrilon and Aptamil in Agostoni et al, 2000, Journal of the American College of Nutrition, Vol. 19, No. 4, 434-438 (33 μmol/l or less in reconstituted ready-to-drink form).

The infant formula is particularly intended for infants that are partially breast-fed by mothers that suffer or have suffered from allergy.

1. A method of fortifying human milk from a lactating woman that suffers from allergy or from a lactating woman that has suffered from allergy, said method comprising the steps of:
a) providing a supplement comprising free L-glutamine and/or free L-asparagine, and
b) adding the supplement provided in step a) to human milk from a lactating woman that suffers from allergy or from a lactating woman that has suffered from allergy, thereby obtaining a fortified human milk.

2. A method of providing infant nutrition to an infant who is at least partially fed with human milk of a lactating woman that suffers from allergy or of a lactating woman that has suffered from allergy, said method comprising the steps of:
i) fortifying infant nutrition with a supplement comprising free L-glutamine and/or free L-asparagine, and
ii) feeding the fortified infant nutrition to the infant.

3. The method according to claim 2, wherein the infant nutrition is a) human milk from a lactating woman that suffers from allergy or from a lactating woman that has suffered from allergy or b) an infant formula.

4. The method according to claim 1, wherein the fortified infant nutrition, in particular the fortified human milk, contains free L-glutamine and/or free L-asparagine in an amount of at least 8.5 mg/100 ml, preferably 8.5 to 100 mg/100 ml.

5. The method according to claim 2, wherein the infant is fed the fortified infant nutrition at least once a day.

6. The method according to claim 1, wherein the supplement comprising free L-glutamine and/or free L-asparagine is in powder form.

7-11. (canceled)

12. An infant formula fortified with free L-glutamine and/or free L-asparagine for use in the treatment or prevention or reduction of the risk of allergy in an infant of a lactating woman that suffers from allergy or of a lactating woman that has suffered from allergy, wherein the infant is at least partially fed with the human milk of the lactating woman.

13. The infant formula for use according to claim 12, wherein the infant formula comprises at least 8.5 mg/100 ml, preferably 8.5 to 100 mg/100 ml of the sum of free L-glutamine and free L-asparagine when reconstituted in water.

14. The infant formula for use according to claim 12, wherein the weight ratio of free L-glutamine to free L-asparagine is 50/1 to 5/1, preferably 40/1 to 7/1.

15. The method according to claim 2, wherein the infant is term and/or has a birth weight appropriate for gestational age.

16. The method according to claim 1, wherein the allergy is selected from hay fever, atopic dermatitis and asthma.

17. The method according to claim 1, wherein the supplement comprises free L-glutamine.

18. The method according to claim 1, wherein the fortified infant formula comprises free L-glutamine.
